(19) United States
(12) Patent Application Publication
(10) Pub. No.: US 2010/0119582 A1
(43) Pub. Date: May 13, 2010

(54) Device for the Treatment and Prevention of Disease, and Methods Related Thereto

(76) Inventors: Lars Boerger, Duesseldorf (DE); Wolfgang Daum, Groton, MA (US)

Correspondence Address: LUCY ELANDJIAN;IP LAW SERVICES, LLC., 450 E. Waterside Drive, Suite 202, Chicago, IL, 60601, US

(21) Appl. No.: 12/625,582

(22) Filed: Nov. 25, 2009

Related U.S. Application Data
(63) Continuation-in-part of application No. 10/784,331, filed on Feb. 23, 2004, now abandoned.
(60) Provisional application No. 60/448,930, filed on Feb. 22, 2003.

(51) Int. Cl. A61F 2/00

(52) U.S. Cl. 424/426

(57) ABSTRACT

Disclosed are implantable devices for delivering a drug into the blood stream of a vessel or into the vessel wall of a subject's body to treat or prevent vascular or cardiovascular disease, such as vascular plaque, cardiovascular plaque, and diseases attributable to inflammation, such as arteriosclerosis, diabetes, rheumatoid arthritis, and Alzheimer's disease. The devices of the present invention comprise a biodegradable matrix that degrades gradually and vanishes over a period of time, and have a ring-like, flag-like, or plaster-like configuration. The flag-like configuration comprises a holding structure and at least one flag. These flags are preferably elastic, and may be constructed from fibers, woven tissue, strings, sheets, or any combination thereof. Disclosed devices may comprise more than one drug, or varying concentrations of the same drug. Also disclosed are methods related thereto.

### CROSS REFERENCE TO RELATED APPLICATIONS

This is a non-provisional application claiming the benefit of and priority to U.S. non-provisional patent application having Ser. No. 10/784,331, filed on Feb. 23, 2004, and U.S. provisional patent application having Ser. No. 60/448,930, filed on Feb. 22, 2003, both of which are incorporated herein by reference.

### TECHNICAL FIELD

The present invention relates generally to implantable devices. Specifically, the invention pertains to an implantable device that releases a drug or pharmaceutical agent to treat or prevent cardiovascular or vascular diseases, and methods related thereto.

### BACKGROUND

Vascular disease is a leading cause of death and disability. In the United States, more than one half of all deaths are due to cardiovascular disease. Arteriosclerosis is the most common form of vascular disease and leads to insufficient blood supply to body organs, which can result in hearts attacks, strokes, and kidney failure.

### 1. Atherosclerosis and Plaques

Atherosclerosis is a form of vascular injury in which the vascular smooth muscle cells in the artery wall undergo hyperproliferation and invade and spread into the inner vessel lining, which can make the vessels susceptible to complete blockage when local blood clotting, referred to as stenosis, occurs. This can lead to death of the tissue served by that artery. In the case of a coronary artery, this blockage can lead to myocardial infarction and death. Atherosclerosis (the most common form of arteriosclerosis, marked by cholesterol-lipid-calcium deposits in arterial linings), “hardening” of the arteries caused by plaques and plaque lesions, is the cause of myocardial infarction (MI). These hard plaques are also referred to as calcified plaques. While some plaques are “hard and solid”, others are “soft and squishy”. It is the soft variety that causes the most concern. This soft plaque is also referred to as “vulnerable plaque” because of its tendency to burst or rupture.

Vulnerable plaques have a lipid-rich core and a thin, macrophage-dense, collagen-poor fibrous cap, and typically cause only mild to moderate stenosis. Factors affecting plaque rupture include mechanical injury, circadian rhythm, inflammation, and infection. Progressive thrombosis and vasospasm may follow plaque rupture. It is believed that physical disruption of such a plaque allows circulating blood coagulation factors to meet with the highly thrombogenic material in the plaque's lipid core, thereby instigating the formation of a potentially occluding and fatal thrombus. Some believe these plaques cause more than 50 percent (“%”) cross-sectional stenosis of the artery.

Mechanical stress and composition of plaques play an important role in plaque disruption. Mechanical forces, including the mere vibration of the heart as it beats, can easily disrupt this plaque. These plaques are classified as either “yellow” or “white”, using coronary angioscopy. Yellow plaques with an increased distensibility and a compensatory enlargement may be mechanically and structurally weak. As a result, mechanical “fatigue,” caused by repetitive stretching, may lead to plaque disruption. Plaques with a high distensibility and a compensatory enlargement may be vulnerable. While a rupturing plaque can lead to a heart attack, most of the time this may not be severe. In fact, it appears that plaques break or rupture all the time, and those that trigger heart attacks are unfortunate exceptions. It is believed that the large plaques visible on angiograms are often the healed-over and more stable remains of small vulnerable plaques.

One of the most important issues pertaining to vulnerable plaques is the fact that vulnerable plaques do not bulge inward. Instead, as a plaque grows, it often protrudes outward, into the wall of the artery, rather than into the channel-lumen where blood flows. On an angiogram, everything can look normal. But when dissected after death, it can be seen that the arteries' walls are thick with plaque which could not yet be seen on an angiogram.

Studies into the composition of vulnerable plaque suggest that the presence of inflammatory cells (and particularly a large lipid core with associated inflammatory cells) is the most powerful predictor of ulceration and/or imminent plaque rupture. In plaque erosion, the endothelium beneath the thrombus is replaced by or interspersed with inflammatory cells.

### 2. Stents and PTCA

Coronary or any peripheral artery blockage can be treated with artery bypass surgery and/or angioplasty. Both procedures may initially appear to be successful, but are in fact undone by the effect of restenosis or the recurrence of stenosis after such a treatment. Restenosis is believed to include hyperproliferation of vascular smooth muscle cells. In particular, one third of patients treated using angioplasty experience restenosis and blockage within 6 months after the procedure. To prevent vessel blockage from restenosis, stents are typically used.

Known stent designs include monofilament wire coil stents (e.g., U.S. Pat. No. 4,969,458); welded metal cages (e.g., U.S. Pat. No. 4,733,665 and U.S. Pat. No. 4,776,337); and, most prominently, thin-walled metal cylinders with axial slots formed around the circumference (e.g., U.S. Pat. No. 4,733,665; U.S. Pat. No. 4,739,762; and U.S. Pat. No. 4,776,337). Known construction materials for use in stents include polymers, organic fabrics and biocompatible metals, such as, stainless steel, gold, silver, tantalum, titanium, and shape memory alloys such as nitinol (i.e., alloys of nickel and titanium).

Of the many problems that may be addressed by stent-based local delivery of beneficial agents, restenosis is one of the most important. Restenosis is a major complication that can arise following vascular interventions, such as angioplasty and the implantation of stents. Simply defined, restenosis is a wound healing process that reduces the vessel lumen diameter by extracellular matrix deposition and vascular smooth muscle cell proliferation, and which may ultimately result in renarrowing or even reocclusion of the lumen. Despite the introduction of improved surgical techniques, devices and pharmaceutical agents, the overall restenosis rate is still reported in the range of 25% to 50% within six to twelve months after an angioplasty procedure. To treat this condition, additional revascularization procedures are frequently required, thereby increasing trauma and risk to the patient, as well as increasing the costs of health care.

Some of the techniques under development to address the problem of restenosis include irradiation of the injury site and the use of conventional stents to deliver a variety of beneficial or pharmaceutical agents to the wall of the traumatized vessel. In the latter case, a conventional stent is frequently surface-coated with a beneficial agent (often a drug-impregnated polymer) and implanted at the angioplasty site. Alternatively, an external drug-impregnated polymer sheath is mounted over the stent and co-deployed in the vessel.

Percutaneous transluminal coronary angioplasty (PTCA) is used as the primary treatment modality in many patients with coronary artery disease. PTCA can relieve myocardial ischemia in patients with coronary artery disease by reducing lumen obstruction and improving coronary flow. Stents and PTCA balloon catheters are usually used for the hard and calcified plaques. There are no solutions on the market yet to treat or prevent the soft or vulnerable plaques.

### 3. Therapeutic Agent.

Therapeutic agents to inhibit restenosis have been used with varying success. Paclitaxel (Taxol®), an antimicrotubule agent isolated from the bark of the western Pacific Yew tree, is especially effective in inhibiting some cancers and is shown to be effective in combating restenosis (e.g., U.S. Pat. No. 5,733,925). Paclitaxel may also prevent thrombus formation. Because systemic administration of paclitaxel can have undesirable side effects, local administration is the preferred mode of treatment.

At least five considerations appear to preclude the use of inhibitory drugs to prevent stenosis resulting from overgrowth of smooth muscle cells.
A. Inhibitory agents may have systemic toxicity that could create an unacceptable level of risk for patients with cardiovascular disease.
B. Inhibitory agents may interfere with vascular wound healing following surgery and that could either delay healing or weaken the structure or elasticity of the newly healed vessel wall.
C. Inhibitory agents killing smooth muscle cells could damage the surrounding endothelium and/or other medial smooth muscle cells. Dead and dying cells also release mitogenic agents that may stimulate additional smooth muscle cell proliferation and exacerbate stenosis.
D. Delivery of therapeutically effective levels of an inhibitory agent may be problematic from several standpoints: namely,
   a. delivery of a large number of molecules into the intercellular spaces between smooth muscle cells may be necessary, i.e., to establish favorable conditions for allowing a therapeutically effective dose of molecules to cross the cell membrane;
   b. directing an inhibitory drug into the proper intracellular compartment, i.e., where its action is exerted may be difficult to control; and,
   c. optimizing the association of the inhibitory drug with its intracellular target (e.g., a ribosome), while minimizing intercellular redistribution of the drug (e.g., to neighbouring cells), may be difficult.
E. Because smooth muscle cell proliferation takes place over several weeks, it would appear that the inhibitory drugs should also be administered over several weeks, perhaps continuously, to produce a beneficial effect.

Hence, local administration of paclitaxel may be more effective when carried out over a longer time period, such as a time period at least matching the normal reaction time of the body to the angioplasty. Local administration of paclitaxel over a period of days or even months may be most effective in inhibiting restenosis. Such a long time period may be successfully provided by a time-release delivery system utilizing a paclitaxel coated stent.

It is now well known that paclitaxel-coated stents reduce neo-intima formation or stenosis.

### 4. Biodegradable Materials

There are many biodegradable polymers in the market that can be useful herein, including those that have proper biomedical approval for use in humans. Biodegradable polymers include starch, cellulose, amylose, polyhydroxybutyrate, lactic or polyactic acid, polybuylenesuccinate, polycaprolactone, aliphatic-aromatic resin, carboxymethylcellulose (CMC) or thermal polyasparatate (TPA).

It has been long known that polylactides comprising poly(L-lactide), poly(D-lactide) or copolymers derived therefrom or with other co-monomers in the form of copolymerizable cyclic esters are usable for human implantable devices.

More recently, several bioabsorbable, biocompatible polymers have been developed for use in medical devices, and approved for such use by the U.S. Food and Drug Administration (FDA). These FDA approved materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials biodegrade in-vivo in a matter of months, although certain more crystalline forms thereof biodegrade more slowly. These materials have been used in orthopedic applications, wound healing applications, and extensively in sutures after processing into fibers. Some of these polymers have also been used in tissue engineering applications.

It has been reported that the polymer hydroxyethyl methacrylate-vinyl pirrolidone is biodegradable and lacks toxicity toward the cells, and hence it is capable of being used for local drug delivery systems (see, Gimeno M J, Garcia-Esteo F, Garcia-Honduvilla N, Bellon J M, Bujan J, Roman J S, Polymer controlled drug delivery system for growth hormone, Drug Deliv 2002 October-December; 9(4):233-7).

### 5. Local Drug Delivery Systems

A newly designed metallic stent containing honeycombed strut elements with inlaid stacked layers of paclitaxel and biodegradable polymer has been demonstrated for instent restenosis prevention (see, Finkelstein et. al. “Local Drug Delivery via a Coronary Stent With Programmable Release Pharmacokinetics”, Circulation. 2003; 107:777). In an in-vitro study, it was shown that manipulation of the layers of biodegradable polymer and drug allowed varying of the initial 24-hour burst release of paclitaxel from about 70% down to about 9%. Late release of drug could be adjusted dependently or independently of early burst release. A biphasic release profile was created by the addition of blank layers of polymer within the stack. In the 30-day porcine coronary model, there was a 70% reduction in late loss, a 28% increase in luminal volume, and a 50% decrease in histological neointimal area compared with bare metal controls. The disadvantage of this approach is that the stent remains in the body after the drug although the bioresorbable coating vanishes, even in the case where a stent is no longer necessary.

Hydrogel-forming polymeric materials have been found to be useful in the formulation of medical devices, such as drug delivery devices. Hydrogel-forming polymers are polymers that are capable of absorbing a substantial amount of water to form elastic or inelastic gels. Many non-toxic hydrogel-forming polymers are known and are easy to formulate. Medical devices incorporating hydrogel-forming polymers offer flexibility in that they can be implantable in liquid or gelled form. Once implanted, the hydrogel-forming polymer absorbs water and thus swells. The release of a pharmacologically active agent incorporated into the device takes place through this gelled matrix via a diffusion mechanism. However, many hydrogel polymers, although biocompatible, are not biodegradable or are not capable of being formed into stable solid devices that can also dissolve over time.

Other reported approaches for delivery of drugs include:
Parenteral delivery of a drug in a biodegradable polymeric matrix to a warm blooded animal, wherein the polymeric matrix comprises a member selected from the group consisting of poly(α-hydroxy acids) and poly(ethylene carbonates) (e.g., U.S. Pat. No. 5,702,717). The drug is released at a controlled rate from the copolymer, which biodegrades into non-toxic products. The degradation rate can be adjusted by proper selection of the poly(α-hydroxy acid).
A transdermal therapeutic system (TTS) for the transcutaneous administration of pergolide over several days (e.g., U.S. Pat. No. 6,461,636). The TTS contains a matrix mass, containing pergolide, taking the form of a layer, which contains a (meth)acrylate copolymer containing ammonio groups or a mixture of a (meth)acrylate copolymer containing amino groups and a (meth)acrylate polymer containing carboxyl groups, 10-50% by weight (hereinafter “wt %”) propylene glycol and up to 5 wt % pergolide.
An expandable medical device, which is stent-like and which has a plurality of elongated struts (e.g., US 2002/0082680). Some of the stent struts include openings in which drugs are integrated for release over time. As with other drug eluting stent embodiments, the stent in this case remains in the body after the drugs have been released, even when the drugs have dispersed and the disease may have been cured.
A device comprising an ocular implant which bio-erodes within the eye environment and thereby gradually releasing the therapeutic agents at the site to be treated until the entire implant eventually erodes without the need for further surgery (e.g., U.S. Pat. No. 4,863,457). Unfortunately, the particular polymer used is not identified. This device is not applicable to cardiovascular diseases.
An erodible device for delivering a drug into the human body, comprising a poly(orthoester) or a poly(orthocarbonate) (e.g., U.S. Pat. No. 4,346,709). It is unclear if the device is intended to erode completely or only partially. Neither a drug that is useful for any vascular disease, nor the use of the device for any vascular disease, is disclosed.
A new protein matrix material for implantable medical devices and implantable drug delivering devices, and methods of making such materials (e.g., US 20020028243). Although the use of this protein based material, which totally disperses, is mentioned for the use of implants, only examples given are those of encapsulated or coated stents, in which only the coating vanishes. This protein based material appears well-suited for growth of cells on and/or within the material matrix, but it is not suitable for rigid implants due to the fragile nature of the protein.

### SUMMARY OF THE INVENTION

In view of the above, there is a need for an implantable drug delivery device that releases a drug over a period of time, and degrades thereafter. There is also a need for methods related to such devices for the treatment or prevention of cardiovascular or vascular diseases.

It is, therefore, an aspect of the present invention to provide an implantable drug delivery device that releases one or more drugs, preferably over a period of time, and vanishes thereafter.

It is also an aspect of the present invention to provide an implantable drug delivery device for the treatment or prevention of cardiovascular or vascular diseases.

It is also an aspect of the present invention to provide a method for treatment or prevention of cardiovascular or vascular diseases via the use of the implantable drug delivery device of the present invention.

The present invention pertains to an implantable drug delivery device comprising a biodegradable matrix, which is coated, loaded and/or filled with at least one drug that is released, preferably gradually, over a period of time, for the treatment or prevention of cardiovascular or vascular diseases, diseases resulting from inflammation, and hard, soft, calcified or vulnerable plaque. The present invention also pertains to methods related to such devices.

The device of the present invention is also suitable for use with patients who have already undergone vascular procedures, for instance a PTCA, or who are classified as high-risk patients due to their family history, their high LDL (low density lipoprotein) or CRP (C-Reactive Protein) levels. The device disclosed herein is useful for local delivery of drugs to treat coronary disease, such as plaques or stenosis. This device may also be used in subjects who already comprise one or more stents; the drug delivery device may be placed in the vessel where the one or more stents are placed, or it may be placed in another vessel of the subject.

The device of the present invention may embody any of various structures, particularly a ring-like structure, a flag-like structure, and a plaster-like structure.

The ring-like structure (hereinafter “RLS”) is deployed in a vessel and fixed to a defined position by gently pushing it outwards against the vessel wall. In one aspect, the RLS comprises a biodegradable matrix material, in which a drug is incorporated and released over time. In another aspect, the RLS comprises a biodegradable matrix coated with a drug or a drug-containing polymer from which the drug dissolves over time. The RLS may also comprise a drug releasing substance. Preferably, the drug, as well as the drug releasing substance, and the biodegradable matrix of the RLS dissolve and vanish over time. The RLS may comprise a circular, elliptical, or any other configuration having circular geometry.

The flag-like structure (hereinafter “FLS”), comprising a holding structure, which may be ring-shaped, and at least one flag, is deployed in a vessel; the holding structure of the FLS is fixed to a defined position in the vessel as it is gently pushed outwards against the vessel wall. In one aspect, the flag(s) of the FLS comprises a biodegradable matrix material and at least one drug, which is released over a period of time. In another aspect, the holding structure of the FLS comprises a biodegradable matrix material and at least one drug, which is released over a period of time. In another aspect, the holding structure and the flag(s) of the FLS comprise a biodegradable matrix material and at least one drug, which is released over a period of time. The drug(s) released from the holding structure may be the same as or different from the drug(s) released from the flag(s); where the drug(s) released is the same, the concentration of the drug released from the holding structure may be the same as or different from the concentration of the drug released from the flag(s). The holding structure and/or the flag(s) of the FLS may also comprise a drug releasing substance. The drug(s), as well as the drug releasing substance, and the biodegradable matrix of the FLS dissolve and vanish over a period of time. Compared to the RLS, the FLS comprises a larger drug-eluting surface and hence can release the drug(s) more quickly.

The plaster-like structure (hereinafter “PLS”) is deployed on the vessel wall. In one aspect, the PLS comprises a biodegradable matrix material and a drug, which is released over a period of time. The PLS may also comprise a drug releasing substance. The drug, as well as the drug releasing substance, and the biodegradable matrix of the PLS dissolve and vanish over a period of time.

The above summary of the present invention is not intended to describe each illustrated aspect or every implementation of the present invention. The figures and the detailed description that follow particularly exemplify these aspects.

### BRIEF DESCRIPTION OF THE DRAWINGS

The invention may be more completely understood in consideration of the following detailed description of various aspects of the invention in connection with the accompanying drawings, in which:

FIG. 1 schematically illustrates an implantable drug delivery device having a RLS configuration, deployed in a manner similar to that of a stent, according to one aspect of the present invention, specifically:

FIG. 1a illustrates a system for deploying the implantable device,

FIG. 1b and FIG. 1c illustrate longitudinal-sectional and cross-sectional views, respectively, of the implantable device being guided with a balloon in the vessel,

FIG. 1d and FIG. 1e illustrate longitudinal-sectional and cross-sectional views respectively, of the implantable device stretching to the vessel as the balloon is expanded,

FIG. 1f and FIG. 1g illustrate longitudinal-sectional and cross-sectional views, respectively, of the implantable device clamped and remaining at the vessel wall;

FIG. 2 schematically illustrates an expandable holding structure of the RLS configuration of the device, according to one aspect of the present invention, specifically:

FIG. 2a illustrates a three-dimensional view of FIG. 1a, and

FIG. 2b illustrates an unfolded view of FIG. 1b;

FIG. 3 schematically illustrates a cross-sectional view of a strap, of a RLS configuration of the device, having a coating on the inner side, specifically:

FIG. 3a illustrates a cross-sectional view of a strap having a drug containing layer, according to one aspect of the present invention,

FIG. 3b illustrates a cross-sectional view of a strap having an etched inner surface, according to one aspect of the present invention,

FIG. 3c illustrates a cross-sectional view of a strap having two drug-containing coatings, according to one aspect of the present invention;

FIG. 4 schematically illustrates another method for deploying an implantable drug delivery device having a RLS configuration, according to one aspect of the present invention, specifically:

FIG. 4a illustrates a system for deploying the implantable device whereby the device is mounted on an expanded balloon,

FIG. 4b illustrates, in longitudinal-sectional view, the system with the balloon in expanded mode,

FIG. 4c illustrates, in longitudinal-sectional view, the system with the balloon in contracted mode,

FIG. 4d and FIG. 4e illustrate, in longitudinal-sectional view, the implantable device mounted on an expanded spring;

FIG. 5 schematically illustrates an implantable drug delivery device having an FLS configuration, placed in a coronary vessel, behind the location where the vessel branches from the ascending aorta, according to one aspect of the present invention;

FIG. 6 schematically illustrates a flag of the FLS configuration of an implantable drug delivery device, specifically:

FIG. 6a illustrates a cross-sectional view of the flag containing woven or twisted fibers, according to one aspect of the present invention,

FIG. 6b illustrates a cross-sectional view of the flag containing unwoven fibers, according to one aspect of the present invention,

FIG. 6c illustrates a three-dimensional view of a tapered fiber, according to one aspect of the present invention;

FIG. 7 schematically illustrates an implantable drug delivery device having a PLS configuration, which is deployed in a manner similar to that of a stent, according to one aspect of the present invention, specifically:

FIG. 7a illustrates a system for deploying the implantable device,

FIG. 7b and FIG. 7c illustrate longitudinal-sectional and cross-sectional views, respectively, of the implantable device being guided with a balloon in the vessel,

FIG. 7d and FIG. 7e illustrate longitudinal-sectional and cross-sectional views, respectively, of the implantable device stretching to the vessel as the balloon is expanded,

FIG. 7f and FIG. 7g illustrate longitudinal-sectional and cross-sectional views, respectively, of the implantable device stuck to and remaining at the vessel wall; and,

FIG. 8 schematically illustrates a cross-sectional view of the matrix material of an implantable drug delivery device comprising coated particles, according to one aspect of the present invention.

While the invention is amenable to various modifications and alternative forms, specifics thereof have been shown by way of example in the drawings and will be described in detail. It should be understood, however, that the intention is not to limit the invention to the particular aspects described. On the contrary, the intention is to cover all modifications, equivalents, and alternatives falling within the spirit and scope of the invention as defined by the appended claims.

### DETAILED DESCRIPTION

The present invention pertains to implantable devices for delivery of a drug into a vessel system of a patient's body, e.g., the cardiovascular or coronary system, to treat the vessel system or parts thereof, or to prevent the vessel system or parts thereof from a disease. The drug may be coated onto one or more surfaces, or incorporated into the matrix material of the holding structure of the device, or any combination thereof. In the case of the FLS, the drug may alternatively or in addition be coated onto one or more surfaces of the one or more flags, or coated onto the fibers and/or other components of the flag(s), or incorporated into the matrix material of the fibers and/or other components of the one or more flags of the device, or any combination thereof. The drug is released from the device over a period of time, preferably gradually, with the rate of release being based on body temperature as well as on the chemical, biochemical, or physical reactions between the device and the blood. Thereafter, the device degrades, and preferably, degrades completely and vanishes from the body, preferably, removed from the body by the body's natural processes.

### 1. DEFINITIONS

The term “biodegradable”, “bioabsorbable”, or “bioresorbable”, as used herein, refers to any material that is in contact with subject's body tissue or fluids and that is susceptible to breakdown to lesser molecular weight components.

The term “diffusion” or “diffuses”, “degradation” or “degrades”, “dissolves”, and “erosion” or “erodes”, as used herein, refers to a process in which the matrix material of the device leaves the subject's body. Preferably, these terms refer to the gradual and complete removal of the device from the body. The physical, chemical or biochemical process between diffusion, degradation, dissolving, and erosion may be different.

The term “drug”, as used herein, refers to a substance or medication used in the diagnosis, treatment, or prevention of a disease, and includes the terms “pharmacologically active agent”, “pharmaceutical active ingredient”, “pharmaceutical agent” and “pharmaceutical composition”.

The term “elution” or “elute”, “diffusion” or “diffuses”, “dissolve”, and “controlled release” or “releases” as used herein, refers to a process in which a drug leaves the drug delivery device. The physical, chemical or biochemical process between elution, diffusion, and dissolving may be different.

The term “flag-like”, as used herein, refers to the construction and mechanical behaviour of the device being similar to a flag, in that the flag of the device is attached at one portion (e.g., at an end or a corner) to the holding structure of the device (similar to a flag that is attached, for example, to a flag pole), and the flag floats in the bloodstream (similar to a flag floating or waving in the air).

The term “matrix”, as used herein, refers to the material environment of the device and describes a material composition of different materials, elements, and etc. The matrix may comprise fibers or particles that are coated with and/or incorporate one or more drugs.

The term “plaque”, as used herein, refers to calcified vascular plaque, vulnerable vascular plaque, hard vascular plaque, soft vascular plaque, or any combination of thereof.

The term “to deploy a device into a vessel”, as used herein, refers to determining a beneficial location for implanting the device, preferably using any type of a radiological imaging modality, introducing the device into the vessel, and placing the device therein, and leaving it at the beneficial location.

It is to be understood that the singular forms of “a”, “an”, and “the”, as used herein and in the appended claims, include plural reference unless the context clearly dictates otherwise.

### 2. IMPLANTABLE DEVICE CONFIGURATIONS

The implantable device of the present invention embodies any of various configurations, particularly a ring-like structure, a flag-like structure, and a plaster-like structure. These devices are designed solely for drug delivery.

A. Ring-Like Structure (RLS)

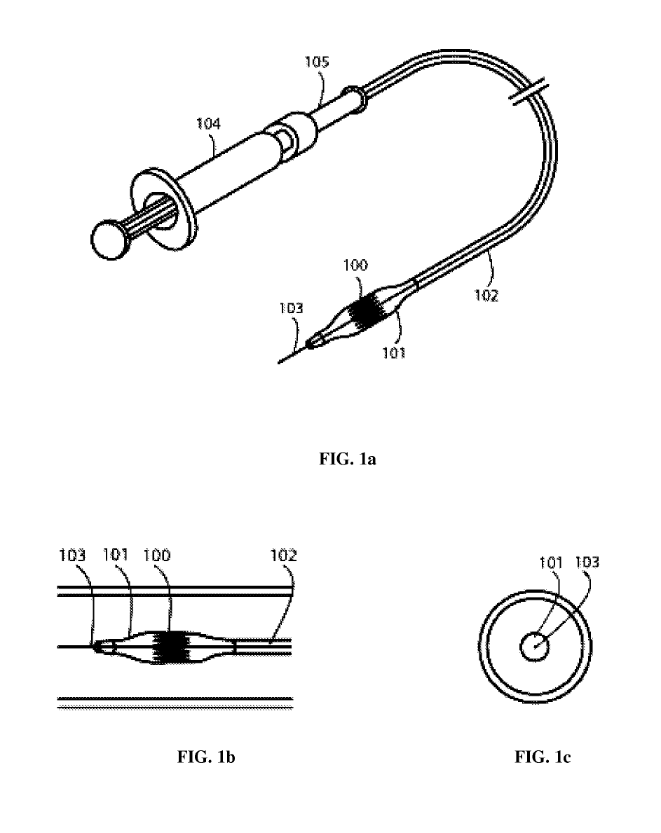

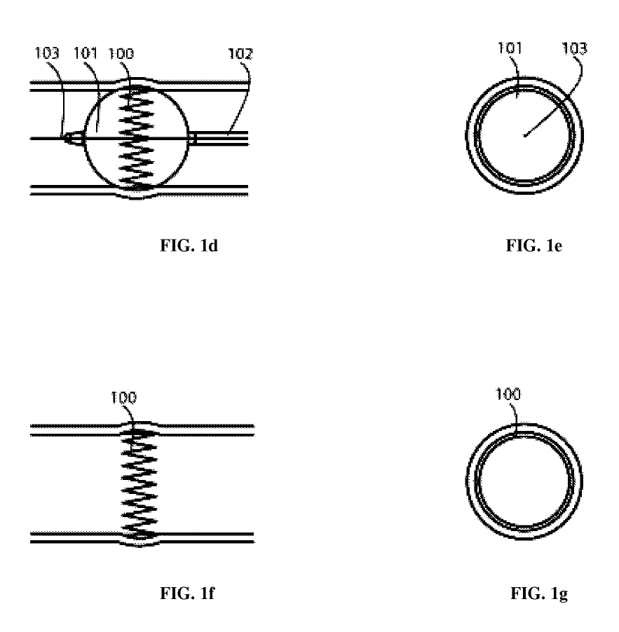

An implantable drug delivery device having a ring-like structure configuration, which can be deployed in a manner similar to a standard state-of-the-art vascular or cardiovascular stent, is demonstrated in FIG. 1. An RLS 100 is mounted on a balloon 101, in a manner similar to mounting a stent, and the balloon 101 is mounted on a catheter system 102, as illustrated in FIG. 1a. The catheter system 102 with balloon 101 is guided with the help of a guide wire 103 into a vessel 106. A syringe 104 is adapted via an adapter 105 onto the catheter system 102 to expand the balloon 101, e.g., with NaCl (sodium-chloride) solution. This procedure is illustrated in FIG. 1b through FIG. 1g. The balloon 101 with RLS 100 is guided with the guide wire 103 in the vessel 106, as illustrated in FIG. 1b and FIG. 1c, under standard imaging modalities, such as ionizing radiation (x-ray), ultrasound (US) or magnetic resonance imaging (MRI). The balloon 101 is then expanded, as illustrated in FIG. 1d and FIG. 1e, and the RLS 100 stretches beyond its elastic limits, thus becoming deformed. Once the balloon 101 is contracted and removed, the RLS 100 will remain in the vessel 106, gently clamping itself outwards against the wall of vessel 106, and thus remaining at that position, as illustrated in FIG. 1f and FIG. 1g.

An implantable device having a ring-like structure (RLS) differs from a stent in that the stent requires more mechanical strength to push against the vessel wall as the stent has to hold open the vessel, while the RLS only has to clamp itself against the vessel wall to stay in place. The purpose of a stent is to hold the vessel open by mechanical strength to prevent the vessel from occlusion, while the purpose of the RLS is to stay in place and release a drug. Although the RLS may be as long as a stent or shaped exactly like a stent, the two devices serve different purposes, and are therefore constructed differently. Because a stent generally has to hold the vessel open over a longer distance of space and a longer duration of time, the stent must be constructed in such a way that it sustains more mechanical force. Because the RLS has to carry a drug to be released into the blood stream or to the vessel wall itself, and to dissolve and vanish thereafter, it does not need to be constructed to sustain a mechanical force of the magnitude required for a stent. Although some stents may comprise drug coatings, typically, the purpose of those coatings is to prevent the stent from re-closing or re-occluding (referred to as instent-restenosis). Whereas, the RLS is utilized to deliver one or more drugs that can have an effect in the blood, on the vessel's inner surface, in the vessel wall further down the blood stream, and/or in the vessel wall at the location of the RLS.

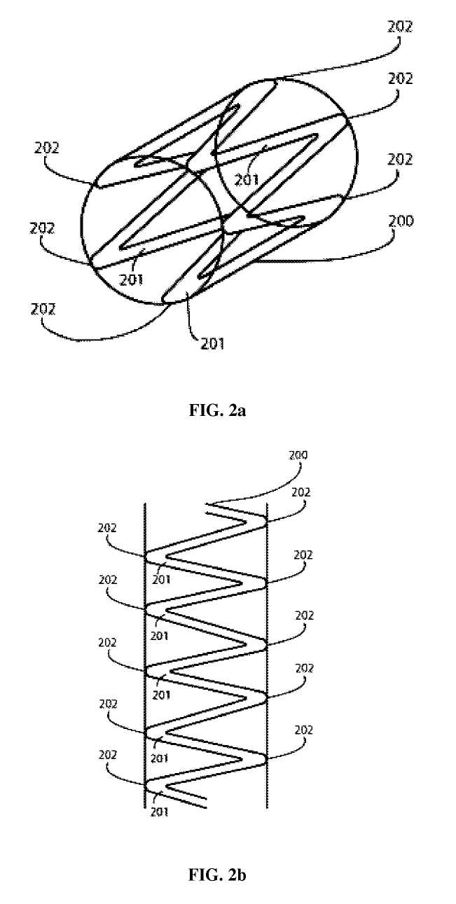

In one aspect of the present invention, the RLS comprises a zigzag expandable structure as illustrated in three-dimension in FIG. 2a. An RLS structure 200 is cut from a tube using a laser, in a manner similar to standard stents. Alternatively, the RLS may be cut from a sheet and glued or welded to yield a tube-like geometry, as illustrated in FIG. 2a. The RLS 200 as unfolded is illustrated in FIG. 2b. During the expansion of the RLS 200, the straps 201 at the edges 202 bend over their elastic limits and become plastically deformed to remain in the expanded geometry. The straps are pieces of the RLS that are deformed beyond their plasticity limits so that the RLS does not bend back. The RLS may comprise one or more straps. Different geometries and designs are also possible for the RLS.

The RLS is preferably constructed from a biodegradable matrix material, one that degrades over time, more preferably one that degrades after a drug coating has dissolved or after the drug incorporated in the matrix has washed out, diffused out, dissolved, or eluted.

In one aspect of the present invention, the RLS is deployed e.g., in a cardiovascular artery or any other vessel proximal to or at the area in which the drug shall be effective. To treat an entire artery, the RLS may be deployed in the artery behind the location where the artery branches from the ascending aorta. The RLS typically has the following dimensions:
unexpanded diameter: in the range of from about 0.5 mm to about 5 mm, preferably, about 1 mm to about 2 mm,
expanded diameter: in the range of from about 2 mm to about 12 mm, preferably, about 3 mm to about 5 mm,
wall thickness: in the range of from about 0.07 mm to about 0.5 mm, preferably, about 0.07 mm to about 0.12 mm,
length: in the range of from about 3 mm to about 20 mm, preferably, about 4 mm to about 6 mm,
strap-width: in the range of from about 0.1 mm to about 5 mm, preferably, about 0.1 mm to about 1 mm.

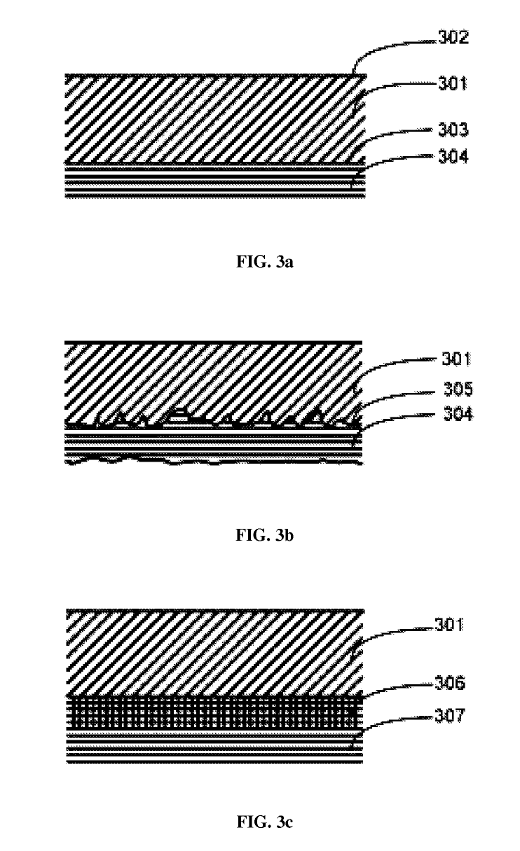

The RLS has basically two surfaces, the outer surface facing the vessel wall and the inner surface facing the lumen or the blood stream in the vessel. In one aspect of the present invention, at least the inner surface of the RLS is coated with a drug. The drug is released by the coating and it dissolves in the blood stream, whereby it is transported downstream the vessel to be effective on sites of the vessel wall or in further vessels distal to the location in which the RLS was deployed. FIG. 3a illustrates a cross-sectional view of a strap 301 of an RLS, having an outer surface 302, an inner surface 303 and a drug containing layer 304, which is coated on the inner surface 303. In one aspect of the invention, the drug-containing layer is coated on the outer surface 302. The drug that is coated on the outer surface 302 and the drug that is coated on the inner surface 303 may be the same drug or may be different drugs.

Un-isotropic chemical or physical etching may increase the roughness of the inner surface 305 of the strap 301 of an RLS, as illustrated in FIG. 3b. This type of etching selectively etches grain-boundaries in the material, giving the surface a larger surface for the coating. There may be more than one drug-containing surface, for example, in FIG. 3c, two drug-containing coatings 306 and 307 are illustrated. The coatings may dissolve at different times, for example, coating 307 may dissolve before or more quickly than coating 306 dissolves. The different dissolving schedules may be due to the intended use of the drug; for example, the purpose of coating 307 may be to treat an acute or severe disease, such as the beginning of a stenosis or vulnerable plaque, while the purpose of coating 306 may be to prevent the vessel from the recurrence of said disease in the near future.

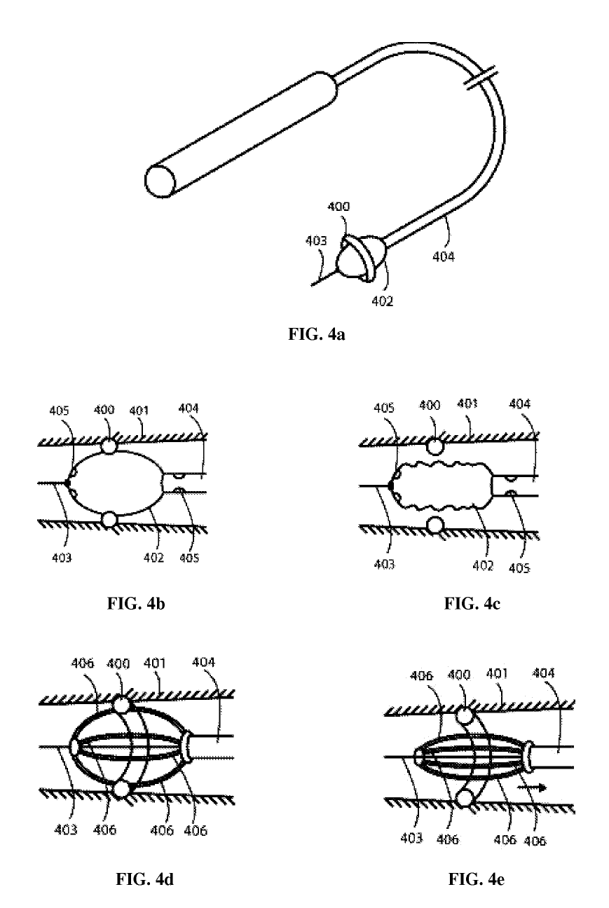

Another method for deploying an RLS in a coronary vessel is illustrated in FIG. 4. A device 400, having a ring-like structure and comprising a ring, is introduced into the vascular system from the external iliac artery, passing into the aorta upstream. The device 400 is then pushed downstream in a desired cardiovascular vessel 401, thus the diameter of the vessel 401 decreases. The intent for this device is to enter the vascular system with a ring in its full dimensions and push it down the cardiovascular artery until the RLS 400 can not be pushed any further, where the diameter of the RLS ring is the same as that of the artery, and the RLS comes to a stop. At this position, the RLS 400 clamps itself into the cross-section of the artery. The introduction of a full size ring having an outer diameter of about 3 mm to about 4 mm into the iliac artery or the aorta is not shown herein, but may be accomplished, e.g., with a needle of appropriate inner diameter.

RLS 400 is mounted on an expanded balloon 402 and is pushed through the vessel with the help of a guide wire 403 and catheter 404. FIG. 4a and FIG. 4b illustrate this process in a longitudinal cross-sectional view. In FIG. 4a, balloon 402 is mounted on the distal part of catheter 404, in an expanded mode. Pass through holes 405 allow blood to flow from the proximal side of the balloon 402 to the distal side of the balloon 402. Once the RLS 400 is clamped into the wall of vessel 401, the balloon 402 is deflated and can be withdrawn from the site, as illustrated in FIG. 4c. The RLS 400 is thus deployed.

The use of a balloon can often be disadvantageous in that blood cannot flow steadily during the time of deployment. FIG. 4d and FIG. 4e illustrate an enhanced system in which the RLS 400 is mounted on an expanded spring 406. When the RLS 400 is clamped in the wall of vessel 401, the spring 406 is released by pulling back the catheter 404, as shown with the arrow in FIG. 4e, and the catheter 404 is withdrawn from the vessel.

A biodegradable RLS may comprise one or more areas or layers of material that degrade, dissolve, elute or vanish over time. These areas or layers may be different and may comprise different drugs, or comprise the same drug but in differing concentrations.

The RLS may have a circular, elliptical, or any other configuration having circular-type geometry.

B. Flag-Like Structure (FLS)

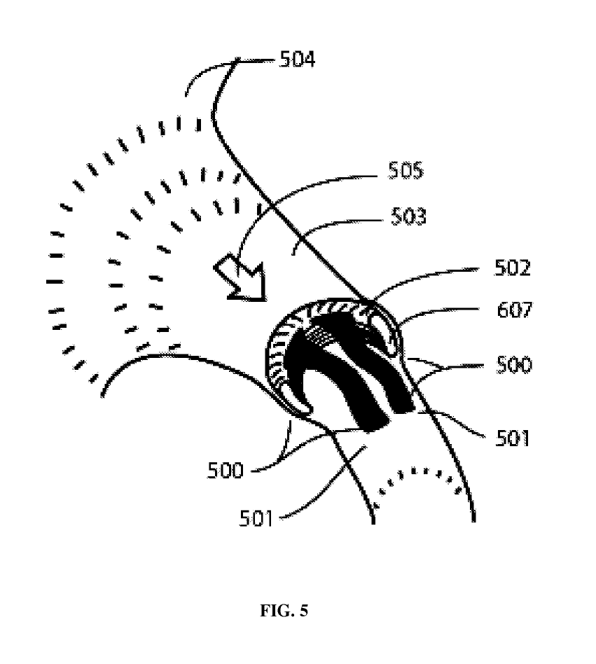

FIG. 5 illustrates an implantable drug delivery device having a flag-like structure configuration 500, comprising one or more flags 501 and a holding structure 502, placed inside of a vessel. The FLS matrix preferably comprises a drug; the drug may be incorporated into the matrix, and/or be coated onto or underneath the matrix material.

Flag(s) 501 of the FLS may be constructed from fibers, woven tissue, strings, sheets, or any combination thereof. Flag(s) 501 are elastic and float in the blood stream, as indicated by arrow 505. The matrix material of the FLS 500 may be any material suitable for use herein, preferably a biodegradable polymer. Flag(s) 501 are attached to the holding structure 502 via any suitable means, such as glue, mechanical clamping or moulding thereto. Flag(s) 501 comprise a length in the range of from about 0.1 mm to about 100 mm, preferably, from about 5 mm to about 20 mm. Flag(s) 501 comprise a width or diameter (depending on the configuration of the flag) in the range of from about 0.1 mm to about 5 mm; the width or diameter of flag(s) 501 may be gradually tapered such that the width or diameter at the proximal end (where the flag 501 is attached to the holding structure 502) is larger than the width or diameter at the distal end. Holding structure 502 gently clamps itself from inside out against the wall of a coronary vessel 503, behind the location where the vessel branches from an ascending aorta 504. Holding structure 502 preferably a comprises a ring configuration, but it may be a stent; it is deployed in a manner similar to that of a balloon, an expandable stent, or an RLS, as described above.

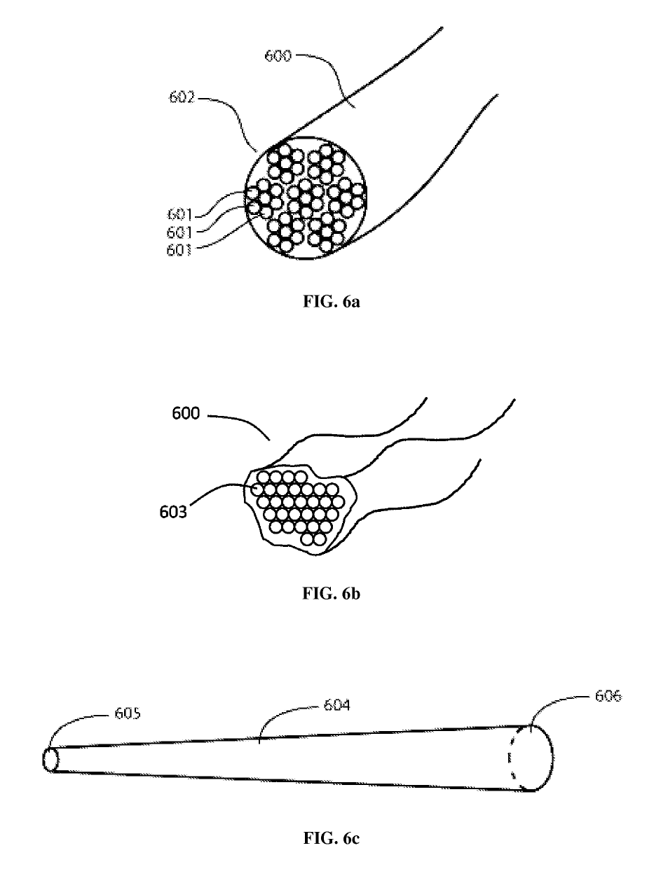

The structure of an FLS flag, wherein individual fibers form a substructure which are combined to yield the overall structure of the flag, is illustrated in FIG. 6a and FIG. 6b. In one aspect of the present invention, individual fibers 601 form a substructure 602, which then can be woven or twisted to yield the overall structure of the flag 600 (FIG. 6a). In another aspect, individual fibers 603 may be unwoven, and lie or float in the bloodstream while remaining attached to substructure 602 (FIG. 6b). In another aspect, individual fibers 604 may be tapered, with a thinner distal portion 605 as compared to a proximal portion 606, as illustrated in FIG. 6c. Similar to the tapered fibers, flags 501 may also be tapered. If such a tapering fiber or flag biologically degrades over time, it will diminish from the distal portion 605, leaving the proximal portion 606 attached to a holding structure 607 (shown in FIG. 5). The tapering design eliminates broken fiber parts or broken flag parts drifting apart from the FLS (which is a likely occurrence with fibers or flags that have that have a constant cross-section over the distance along their longitudinal axis), and thus leaving the principle structure of the FLS intact.

In one aspect of the present device, the flags are coated with a drug by dipping them at least once into the drug. The flags have, advantageously, a high ratio of surface to volume, referred to as aspect ratio, which enables the flags to be coated with a large amount of drug. The high aspect ratio can also be useful when faster dilution of the drug is desired, meaning, the greater the surface area, the faster the drug dilution.

Different flags of the present device may comprise the same drug, or different drugs, or different concentrations of the same drug. In one aspect of the present device, one flag comprises paclitaxel while another flag comprises another drug suitable for use herein. In another aspect, one flag comprises X concentration of paclitaxel, while another flag comprises Y concentration of paclitaxel. In another aspect, a given flag comprises different concentrations of the same drug, as the different fiber or tissue or sheet or string components of that flag comprise the different drug concentrations. In another aspect, a given flag comprises different drugs, as the different fiber or tissue or sheet or string components of that flag comprise the different drugs. Further, the holding structure of the FLS may comprise the same drug, or different drugs, or different concentrations of the same drug than that of the flags of the device. Different drugs or different concentrations of the same drug may be desired in order to treat different diseases or different aspects of a disease. For example, some flags may contain a drug that treats calcified plaque and while other flag(s) may contain a drug that treats vulnerable plaque. Further, different drugs may be utilized based on the desired effect, such as elution rate, as some drugs elute more quickly than others.

C. Plaster-Like Structure (PLS)

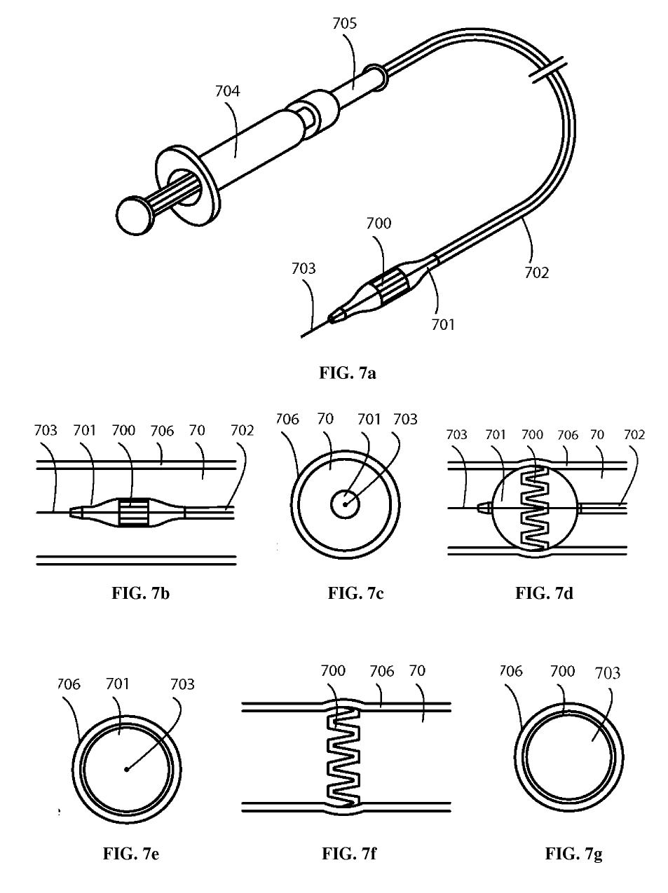

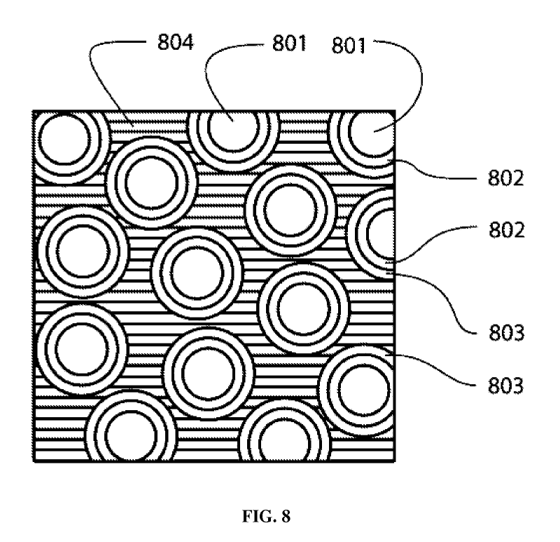

FIG. 7 demonstrates a plaster-like structure 700, which can be deployed in a manner similar to a standard state of the art vascular or cardiovascular stent. The plaster material of the PLS 700 comprises a glue to facilitate its attachment to the vessel wall. PLS 700 is mounted on a balloon 701, in a manner similar to a standard stent, with the balloon 701 being mounted on a catheter system 702. The catheter system 702 with balloon 701 is guided with the help of a guide wire 703 into and through a vessel 706. A syringe 704 is adapted via an adapter 705 onto the catheter system 702 to expand the balloon 701, e.g., with NaCl (sodium-chloride) solution. This procedure is illustrated in FIG. 7b through FIG. 7g. The balloon 701 with PLS 700 is guided with the guide wire 703 in the vessel 704, as illustrated in FIG. 7b and FIG. 7c. The balloon 701 is then expanded, as illustrated in FIG. 7d and FIG. 7e, whereby the PLS 700 stretches and sticks, via the glue, to the wall of vessel 706. The glue does not stick below a threshold temperature, e.g., 40° C., and will melt, but will stick above said threshold temperature. Once the balloon 701 is contracted by releasing the pressure from the balloon, the PLS 700 will remain at the wall of vessel 706 in that position, as illustrated in FIG. 7f and FIG. 7g.

In another aspect of the present device, the matrix material of the PLS is an elastic material and will harden, once the temperature of the NaCl solution in the balloon is raised above a defined threshold temperature. It must be noted that the RLS, as well as the holding structure, e.g., a ring, of the FLS, may be deployed in the same manner, via hardening a material by temperature change. To reach the threshold temperature in the NaCl solution, an energy source heating, e.g., a heating means or heating element, may be used within the balloon, or a warmed NaCl solution may be pumped into the balloon excorporeally. The energy source may be a resistive electrical wire, a laser (such as a diode laser) or laser fiber, a radio frequency or microwave source, or a chemical reaction.

The PLS is constructed from a biodegradable polymer, and hence will dissolve over a period of time. Even in the late phase of the bioresorption of the plaster, no parts loosen from the vessel wall to drift into the blood stream and lead to an occlusion of the vessel because any remaining unresorbed fragments typically remain glued to the vessel wall. This is an advantage of using a PLS. The PLS may comprise one or more areas or layers, which may be different, and which comprise different drugs, or the same drug but in differing concentrations.

### 3. DEVICE COMPOSITION

The implantable drug delivery device of the present invention comprises a biodegradable matrix and, preferably, at least one drug. The drug(s) may be incorporated into the biodegradable matrix via any suitable means, including as layers, or may be coated onto at least one surface of the device, or a combination thereof.

In one aspect of the present invention, the biodegradable matrix comprises drug-coated particles. Because not every drug can easily be mixed into the polymer, it may be more efficient to coat the drug on particles and mix these coated particles into the polymer matrix. The particles may comprise the same polymer that the polymer matrix comprises, or they may be selected from different materials, such as iron-oxide (Fe₃O₄), titanium, titaniumalloys, titaniumoxide (TiO₂), manganese oxide, magnesiumoxide, palladiumoxide, or palladiumcobalt. Each particle may also be coated with a binding-layer, which binds the drug to the particle. Such a binding coating may comprise dextran, any sugar based substance, starch, chitosan, agarose or albumin.

In one aspect of the present invention, particles are coated with synthetic polymers, such as poly(lactic acid), poly(ethylene imine), or poly(alkylcyanoacrylate). Typical particle size ranges from about 40 nanometers (“nm”) to about 1 micrometer (“μm”), preferably from about 100 nm to about 400 nm. The smaller the particle, the better it will be “digested” or removed by the body's metabolism. The thickness of a typical binding layer is in the range of from about 1 nm to about 20 nm. Other materials that may be incorporated into the matrix which are not considered polymers, but provide enhanced features include, but are not limited to, ceramics, bioceramics, bioglasses, glass-ceramics, resin cement, resin fill; more specifically, glass ionomer, hydroxyapatite, calcium sulfate, tricalcium phosphate, calcium phosphate salts, alginate, carbon, and alloys, such as cobalt-based, galvanic-based, stainless steel-based, titanium-based, zirconium oxide, zirconia, aluminum-based, vanadium-based, molybdenum-based, nickel-based, iron-based, and zinc-based alloys (e.g., zinc phosphate, and zinc polycarboxylate).

In one aspect of the present invention, the particles are selected to change the contrast in a radiologic imaging system, such as x-ray (fluoroscopy, angiography, CT, etc.), magnetic resonance imaging (MRI), ultrasound (US) or gamma imaging, such as positron emission tomography (PET). For example, iron oxide (Fe₃O₄) changes the magnetic field around itself and hence lowers the T₁ and T₂ signals in MRI and is mostly seen as black spot. Fe₃O₄ also absorbs x-rays and changes the contrast in x-ray based techniques. Radioactive isotopes, such as ⁹⁰Y, ¹³³Xe, ^{81m}Kr, ¹¹¹In, ^{133m}In, or ²⁰¹Th may be inserted into the mixture to render the device imageable under radioactivity detectors. Gd-DTPA contrast media or gadolinium ions may be inserted into the mixture to render the device MR visible; barium contrast media or barium ions would render the device x-ray visible, and small bubble filled with CO₂ would render the device visible for ultrasound.

One advantage of using a polymer-particle composition for constructing an implantable device of the present invention is that this technique allows the use of polymers, proteins, elastins, or collagens. Typically, these materials, due to their mechanical instability or fast dilution characteristic, are not able to form a solid device with long lasting dilution characteristics. In the device of the present invention, the polymers, proteins or collagens form the binding network between the drug-coated particles. A biocompatible protein for use herein may be naturally occurring or synthetic (including genetically engineered proteins). Naturally occurring proteins include, but are not limited to, elastin, collagen, albumin, keratin, fibronectin, silk, silk fibrin, actin, myosin, fibrinogen, thrombin, aprotinin, antithrombin III, and any other biocompatible natural protein. Specific examples of preferred synthetic proteins for use in the device of the present invention include those commercially available under the nomenclature “ELP”, “SLP”, “CLP”, “SLPL”, “SLPF” and “SELP” (from Protein Polymer Technologies, Inc. San Diego, Calif.). ELP's, SLP's, CLP's, SLPL's, SLPF's and SELP's are families of genetically engineered protein polymers consisting of silk-like blocks, elastin-like blocks, collagen-like blocks, laminin-like blocks, fibronectin-like blocks and the combination of silk-like and elastin-like blocks, respectively. The ELP's, SLP's, CLP's, SLPL's, SLPF's and SELP's are produced in various block lengths and compositional ratios. Generally, blocks include groups of repeating amino acids making up a peptide sequence that occurs in a protein.

The force binding the drug to the particle or the drug to the particle coating may be achieved through intra- and inter-molecular forces (i.e., ionic, dipole-dipole, such as hydrogen bonding, London dispersion, hydrophobic, etc.).

One of the problems associated with the use of drug delivery implants is the exposure of the patient to risk of infection and other medical problems, such as pain and inflammation. To overcome this problem, in one aspect of the present invention, the device is designed to comprise a combination of depolymerized chitosan and a drug, which may be ionically bonded to each other.

Additionally, hydrophobic substances, such as lipids, may be incorporated into the biodegradable matrix of the present device to extend the duration of drug release, while hydrophilic polar additives, such as salts and amino acids, may be added to facilitate, i.e., shorten the duration of, drug release. Exemplary hydrophobic substances for use herein include lipids, e.g., tristeafin, ethyl stearate, phosphotidycholine, polyethylene glycol (PEG); fatty acids, e.g., sebacic acid erucic acid; any combinations of these, and the like.

The controlled release of a drug in a drug delivery device is partially attributed to the homogenous distribution of the pharmacologically active agent(s) throughout the drug delivery device. This homogenous distribution provides for a more systematic, sustainable and consistent release of the pharmacologically active agent(s) by gradual degradation of the device matrix or diffusion of the pharmacologically active agent(s) out of the device. As a result, the release characteristics of the pharmacologically active agent(s) from the device material and/or device are enhanced.

FIG. 8 illustrates a material matrix of a device that comprises one or more coated particles 801. In this particular aspect, the particles are perfectly spherical shaped and all have the same diameter; the shape and dimensions of the particles may be different for other material matrices. Particle(s) 801 are coated with a binding material 802, which binds the particle(s) 801 to a drug 803, which is coated onto the binding layer 802. The coated particle(s) 801 are incorporated into a biodegradable matrix 804. In this particular case, the matrix 804 comprises elastin and hydroxapatite, which resorb in 30 days. The resorbtion rate of this composition over a period of time depends on the inter-particle average distance, which determines how quickly the body fluids can reach the matrix composition to absorb it. In one aspect, the drug layer is paclitaxel. Some of the particles may have a third layer on top of the drug layer 803, wherein this third layer comprises a slow resorbing material to extend the time of drug elution of the device. In one aspect, the binding layer 802 is dextran. Typically, the thickness of any of the layers ranges from about 5 nm to about 100 nm, preferably from about 20 nm to about 30 nm. In one aspect, the particle(s) 801 comprise iron-oxide and have a diameter of about 500 nm. The particle size is selected to ensure that particle(s) 801 can pass through the extra-cellular space when they loosen and dissolve from the device, and are removed via digestion in the body's metabolism.

Each of the RLS, FLS and PLS configurations of the present device may also comprise a suitable drug releasing substance, which along with the drug, dissolves and vanishes from the body over a period of time. Each of the device configurations degrades, preferably gradually over a period of time, until it completely vanishes.

### 4. MATRIX MATERIAL

The matrix of the implantable device of the present invention preferably comprises a biodegradable material. The matrix material may be a polymeric material, a non-polymeric organic material, a metallic material, or any combination thereof.

The biodegradable matrix of the present invention may comprise one or more biodegradable microparticles that provide greater strength to the device. The microparticles may comprise a metal, a plastic, a ceramic, or a combination thereof. These microparticles are so small that they are removed from the body in a natural way, in which the body's metabolism detects and removes unfamiliar or exotic substances. The mixture may be clustered together with an oil-in-water emulsion.

A. Biodegradable Polymer Matrix

There are various biodegradable materials on the market suitable for use herein. Polymeric materials preferable for use as a matrix for the drug delivery device of the present invention include, but are not limited to, a poly(α-hydroxy acid), the copolymers polylactides poly(L-lactide) or poly(D-lactidepoly) or copolymers derived therefrom, such as poly(L-lactide-co-D,L-lactide), poly(L-lactide-co-meso-lactide), poly(L-lactide-co-glycolide), poly(L-lactide-co-trimethylene carbonate), poly(L-lactide-co-ε.-caprolactone), poly(D,L-lactide-co-meso-lactide), poly(D,L-lactide-co-glycolide), poly(D,L-lactide-co-trimethylene carbonate), poly(D,L-lactide-co-ε-caprolactone), poly(meso-lactide-co-glycolide), poly(ethylene carbonate), poly(meso-lactide-co-trimethylene carbonate), poly(meso-lactide-co ε-caprolactone), poly(glycolide-co-trimethylene carbonate), poly(glycolide-co ε-caprolactone), and mixtures thereof. These materials may be purchased as resomers (for example, from Boehringer Ingelheim GmbH, Ingelheim, Germany).

Other examples of biodegradable and/or biocompatible polymeric materials suitable for use herein include, but are not limited to, epoxies, polyesters, acrylics, nylons, silicones, polyanhydride, polyurethane, polycarbonate, poly(tetrafluoroethylene) (PTFE), polyethylene oxide, polycaprolactone, polyethylene glycol, poly(vinyl chloride), polylactic acid, polyglycolic acid, sebacic acid, polypropylene oxide, poly(alkylene)glycol, polyoxyethylene, polyvinyl alcohol (PVA), polymethyl methacrylate, 2-hydroxyethyl methacrylate (HEMA), 1,3-bis(carboxyphenoxy)propane, poly(ethylene oxide) (PEO), polyhydroxybutyrate (PHB), phosphatidylcholine, triglycerides, poly ortho esters, polyhydroxyvalerate (PHV), poly (amino acids), polycynoacrylates, polyphophazenes, polysulfone, polyamine, poly (amido amines), flexible fluoropolymer, isobutyl-based isopropyl styrene, vinyl pyrrolidone, cellulose acetate dibutyrate, silicone rubber, copolymers thereof, and the like.

In one aspect of the present invention, the device is constructed from an elastomeric material, wherein the elastomeric material is a siloxane-based elastomer comprising 3,3,3-trifluoropropyl groups attached to the Si-atoms of the siloxane units, and wherein the elastomer comprises either (i) a mixture comprising a) a non-fluorosubstituted siloxane-based polymer and b) a fluoro-substituted siloxane-based polymer, said polymer comprising 3,3,3-trifluoropropyl groups attached to the Si-atoms of the siloxane units; or (ii) a single siloxane-based polymer comprising 3,3,3-trifluoropropyl groups attached to the Si-atoms of the siloxane units, wherein said polymer or mixture of polymers are cross-linked to form the elastomer.

In an aspect of the present invention, the polymeric matrix may comprise a copolymer of (i) a (meth)acrylate copolymer containing ammonio groups, or (ii) a mixture of a (meth)acrylate copolymer containing amino groups and a (meth)acrylate polymer containing carboxyl groups.

In one aspect of the present invention, the polymeric matrix may comprise a polyurethane elastomeric composition that comprises a soft segment derived from at least one polysiloxane macrodiol and at least one polyether and/or polycarbonate macrodiol. The polyurethane elastomeric composition comprises a soft segment derived from about 60 wt % to about 98 wt % of at least one polysiloxane macrodiol and about 2 wt % to about 40 wt % of at least one polyether and/or polycarbonate macrodiol.

In one aspect of the present invention, the polymeric matrix material biodegrades into non-toxic products. The degradation rate may be adjusted by proper selection of the polymeric material, particularly to provide control over the release rate of the drug(s) incorporated into or coated onto the matrix material.

In one aspect of the present invention, the RLS or the holding structure of the FLS may comprise one or more biodegradable, elastic shape-memory materials. The transition from the temporary to the permanent shape of a thermally induced shape-memory material is initiated by an external stimulus, such as a temperature increase above the switching transition temperature Tₜᵣₐₙₛ of the material. All of these materials are non-degradable in physiological environments and many lack biocompatibility or compliance in mechanical properties. Polymeric materials that are designed to exhibit a thermally induced shape-memory effect require two components on the molecular level: cross-links to determine the permanent shape and switching segments with Tₜᵣₐₙₛ to fix the temporary shape. Above Tₜᵣₐₙₛ, the permanent shape may be deformed by application of an external stress. After cooling below Tₜᵣₐₙₛ and the subsequent release of the external stress, the temporary shape is obtained. Hence, instead of using heat to harden a matrix material of the RLS or FLS, a shape-memory effect approach may be utilized.

B. Biodegradable Metal Matrix

It is known that certain metal alloys are biocompatible and bioresorbable. Such alloys comprise manganese in which lithium is incorporated at about 0.5 wt % to about 20 wt %.

Other metallic materials suitable for use herein include any other biocompatible and biodegradable alloy.

The matrix material of the device disclosed herein may comprise metallic alloys that exhibit shape-memory effect. The shape-memory effect is due to a martensitic phase transition.

C. Non-Polymeric Organic Matrix

There are various non-polymeric organic material useful herein as a matric material. Examples of such non-polymeric biodegradable and/or biocompatible organic materials include, but are not limited to, fibrin, graphite, and lipids.

In one aspect of the present invention, the bioresorbable matrix material is hydroxyapatite (also referred to as hydroxylapatite).

### 5. DRUGS

The device of the present invention comprises one or more drugs for the treatment or prevention of cardiovascular or vascular diseases, such as calcified or vulnerable plaque, and arteriosclerosis.

The drug may be mixed into the matrix material on a molecular or small droplet basis. The size of each droplet ranges from about 10 μm to about 100 μm. These droplets work as little drug depots and open to release the drug when the material of the matrix degrades and vanishes over time. In one aspect, Zyn-Linkers are used to modify the delivery of the drug. Zyn-Linkers are small molecules, which, when chemically coupled to one or more therapeutic agents, anchor them at target sites in the body and release the therapeutic agents at controlled rates over long periods, and thereby reducing the number of required doses and decreasing the side effects of the therapeutic agents.

In one aspect of the present invention, the drug delivery device comprises paclitaxel (a mitotic inhibitor, used in cancer chemotherapy). Other drugs useful herein include dexamethasone (a cortico steroid), rapamicine, tacrolimus, polymer-based copper nitric oxide from S-nitrosoglutathione, and 17-beta-estradiol.

As there are many diseases that are related to inflammation, the present device may comprise one or more drugs for treating or preventing inflammation, particularly in relation to the treatment or prevention of vascular or cardiovascular diseases, rheumatoid arthritis, diabetes, or Alzheimer's disease. Drugs useful herein for preventing or treating inflammation include, but are not limited to, bevacizumab (Avastin®, from Genentech Inc., San Francisco, Calif.), bortezomib (Velcade®, from Millenium Pharmaceuticals, Inc., Cambridge, Mass.), aspirin, statins, beta blockers, and angiotensin converting enzyme (hereinafter “ACE”) inhibitors.

Other drugs suitable for use herein are listed in Table I.

TABLE I
DRUGS SUITABLE FOR USE IN THE PRESENT INVENTION

| Drug | Description |
|---|---|
| Adenosine triphosphate | Anti-arrhythmic, first line drug used for termination of Supraventricular |
| (ATP) | Tachycardias (SVT) involving the AV node or the accessory pathways |
| | (WPW). It can also block the AV node transiently to facilitate the |
| | interpretation of the surface ECG. |
| Alteplace tPA (Tissue | Thrombolytic. Used for lysis of clot inside the coronary vessels in acute |
| Plasminogen Activator) | myocardial infarction; it can also be used for treating pulmonary embolism |
| (Activase ® Genentech) | |
| Amlodipine | Calcium Channel Blocker, 2nd generation. Used for treatment of |
| | hypertension, ischemic heart disease and angina. |
| Amiodarone | Class III anti-arrhythmic. Used for terminating and preventing |
| | supraventricular arrhythmias (SVT) including atrial fibrillation and |
| | ventricular arrhythmias (VT). |
| Anistreplase (APSAC: | Thrombolytic. Used for lysis of clot in the coronary vessels in acute |
| Acylated Plasminogen | myocardial infarction. |
| Streptokinase Complex) | |
| Aspirin (acetylsalicylic | Analgesic. Used also for reducing risk of myocardial infarction and risk of |
| acid) | death after infarction or angina. Also used for reducing risk of |
| | thromboembolism in high risk patients. |
| Atenolol | Beta Blocker. Used for treatment of hypertension, ischemic heart disease, |
| | angina, post myocardial infarction, and heart failure. |
| Atropine | Anti-cholinergic. Used for treatment of bradycardia and heart blockage. |
| Abciximab (ReoPro ®, | A new glycoprotein IIb/IIIa receptor antagonist. Used for complicated |
| Eli Lilly and Company) | PTCA/PTCS procedures; also studied for use in unstable angina and acute |
| | myocardial infarction. |
| Captopril | ACE inhibitor. Used for treatment of hypertension, heart failure and post |
| | myocardial infarction remodelling. |
| Carvedilol | Alpha & Beta Blocker with vasodilator activity. Used for treatment of |
| | congestive heart failure. Start at low dose and titrate up slowly. New |
| | studies show that it reduces mortality in Class II-IV heart failure patients. |
| Celecoxib (Celebrex ®, | Used to treat inflammation. |
| Pfizer, Inc.) | |
| Chlorothiazide | Thiazide. Used for treatment of hypertension and heart failure. |
| Cholestyramine | Bile acid sequestrant. Used for treatment of hyperlipidaemia. |
| Clofibrate | Fibric acid derivative. Used for treatment of hyperlipideamia. |
| Clopidrogel | A new anti-platelet (acts on ADP receptor) with action similar to |
| | ticlodipine. Used for angina, PTCA/S procedures and strokes. New studies |
| | show that it may be useful for unstable angina and myocardial infarction. |
| Digoxin | Digitalis. Used for the control of ventricular rate in atrial fibrillation, heart |
| | failure and PAF. |
| Dipyridamole | Antiplatelet. Used for prevention of thromboembolic disease, cardiac |
| | valvular replacement, and stenting. |
| Disopyramide | Class Ia anti-arrhythmic. Used for treatment of atrial and ventricular |
| | arrhythmias. |
| Dobutamine | Inotopic agent. Used for blood pressure support, and hypotension. |
| Dofetilide | Used for treatment of AF and restoration of normal cardiac rhythm. |
| Dopamine | Inotopic agent. Used for blood pressure support, hypotension, and renal |
| | vascular perfusion (low dose). |
| Enalapril | ACE inhibitor. Used for treatment of hypertension, heart failure and post |
| | myocardial infarction remodelling. |
| Epinephrine | Vasopressor. Used for treatment of hypotension and shock, ventricular |
| | fibrillation, asystole, cardiac arrest, bradycardia, and anaphylactic shock. |
| Felodipine | Calcium Channel Blocker. Used for treatment of hypertension, ischemic |
| | heart disease and angina. |
| Flecainide | Class Ic anti-arrhythmic. Used for treatment of atrial and ventricular |
| (Tambocor ®, 3M | arrhythmias. |
| Pharmaceuticals) | |
| Furosemide | Loop diuretic. Used for treatment of hypertension and heart failure. |
| Heparin | Anti-coagulant. Used for treatment of deep vein thrombosis, pulmonary |
| | embolism, acute myocardial infarction, unstable angina, and peripheral |
| | vessel embolism. |
| Heparin | Anti-coagulant. Used for prophylaxis of deep vein thrombosis and |
| | pulmonary embolism. Also used after PTCA/S. |
| Hydralazine | Direct vasodilator. Used for treatment of malignant hypertension, heart |
| | failure, pre-eclampsia, and eclampsia. |
| Ibutilide (Corvert ®, | Class III anti-arrhythmic. Preparation for acute conversion of atrial |
| Pharmacia & Upjohn | fibrillation or flutter. |
| Company) | |
| Isosorbide dinitrate | Nitrate. Used for treatment of angina and ischemic heart disease. |
| Labetalol | Alpha and Beta Blocker. Used for treatment of hypertension, |
| | pheochromocytoma and dissecting aortic aneurysm. |
| Lidocaine | Class Ib anti-arrhythmic. Used for treatment of ventricular arrhythmic |
| | fibrillation. |
| Lisinopril | ACE inhibitor. Used for treatment of hypertension, heart failure and post |
| | myocardial infarction remodelling. |
| Losartan (Cozaar ®, | Ang II receptor antagonist. Used for treatment of hypertension, may also |
| Merck & Co., Inc) | be used for heart failure. |
| Lovastatin | HMGCoA reductase inhibitor. Used for treatment of hyperlipidemia. |
| Methyldopa | Alpha Blocker (central). Used for treatment of hypertension. |
| Metoprolol | Beta-1-selective Blocker. Used for treatment of hypertension, ischemic |
| | heart disease and post myocardial infarction decrease in mortality. |
| Minoxidil | Direct vasodilator. Used for treatment of hypertension and heart failure. |
| Nifedipine | Calcium Channel Blocker. Used for treatment of hypertension, ischemic |
| | heart disease and angina. |
| Nimodipine | Calcium Channel Blocker. Used for treatment of hypertension, ischemic |
| | heart disease and angina. |
| Nitropusside | Direct vasodilator. Used for treatment of hypertension, heart failure and |
| | dissecting aorta aneurysm. |
| Pravastatin | HMGCoA reductase inhibitor. Used for treatment of hyperlipidemia. |
| Procainamide | Class Ia anti-arrhythmic. Used for treatment of atrial and ventricular |
| | arrhythmias. |
| Propranolol | Beta Blocker. Used for treatment of hypertension, ischemic heart disease, |
| | angina, post myocardial infarction, and heart failure. |
| Protamine | Heparin antagonist. Used for reversal of heparin anticoagulation and |
| | treatment of overdose. |
| Simvastatin | HMGCoA reductase inhibitor. Used for treatment of hyperlipidemia. |
| Sotalol | Class II and III anti-arrhythmic. Used for treatment of supraventricular |
| | arrhythmia and ventricular arrhythmia. |
| Spironolactone | Diuretic. Used for the treatment of heart failure and fluid retention due to |
| (Aldactone ®, | cirrhosis of liver. Recent study (RALES) showed that spironolactone is |
| Pharmacia & Upjohn | useful for heart failure patients. |
| Company) | |
| Streptokinase | Thrombolytic. Used for treatment of acute myocardial infarction (onset of |
| | chest pain less than 12 hours) and pulmonary embolism. |
| Ticlodipine | Anti-platelet agent. Used for stroke prevention and thromboembolic |
| | disease, also used for PTCA and stenting procedure. |
| Urokinase | Thrombolytic. Used for treatment of acute myocardial infarction (onset of |
| | chest pain less than 12 hours) and pulmonary embolism. |
| Verapamil | Calcium Channel Blocker. Used for treatment of hypertension, angina and |
| | atrial arrhythmias. |
| Warfarin | Anti-coagulant. Used for prophylaxis and treatment of thromboembolic |
| | disease, and pulmonary embolism. |

Additional drugs suitable for use herein can be found in “Today in Cardiology”, January 2003 edition, pages 15 to 17 [published by SLACK Inc., 6900 Grove Road, Thorofare, N.J. 08086 USA], said drugs are incorporated herein by reference.

Lactate metal salts, aminoguanidinyl- and alkoxyguanidinyl-substituted phenyl acetamides, 7-oxo-pyridopyrimidines (II), and squaric acid derivatives may also be suitable for use herein. Lactate metal salt, in particular an L-lactate, may also be used for the treatment of arteriosclerosis and/or for the prophylaxis or treatment of diseases caused by arteriosclerosis. Aminoguanidinyl- and alkoxyguanidinyl-substituted phenyl acetamides may be used as protease inhibitors. 7-oxo-pyridopyrimidines (II) may be used as an anti-inflammatory drug. Squaric acid derivatives are able to inhibit the binding of integrins to their ligands and thus are useful in the prophylaxis and treatment of immune of inflammatory disorders, or disorders involving the inappropriate growth or migration of cells.

By reducing LDL cholesterol or other lipids, plaque build-up may be prevented or even reduced. And, within a few months of treatment, plaques may be stabilized. Numerous studies have demonstrated that lowering cholesterol can reduce the risk of heart attack and death in people at high risk of a heart attack. The following types of drugs, including resins, fibrates, niacin or statins, are useful herein for lowering cholesterol.

Resins: Cholestyramine (Questran®) and colestipol (Colestid®, Pharmacia & Upjohn Company)—each lowers cholesterol levels indirectly by binding with bile acids in the intestinal tract. Bile acids are produced in the liver from cholesterol and are needed for food digestion. By tying up bile acids, the drugs prompt the liver to produce more bile acids. Because the liver uses cholesterol to make the acids, less cholesterol is available to reach the bloodstream.

Fibrates (also referred to as fibric acid derivatives): This class of drugs regulates blood serum lipids. Fibrates are particularly useful for lowering triglyceride levels and increasing the levels of HDL (‘good cholesterol’). They work by reducing triglyceride production and removing triglycerides from circulation. Gemfibrozil (Lopid®, Pfizer, Inc.), fenofibrate (Tricor®, Abbott Laboratories Company), and bezafibrate (Bezalip, Hoffmann-La Roche Ltd.) are exemplary fibrates.

Niacin (also referred to as nicotinic acid): Large doses of niacin, a vitamin, also can lower triglycerides. In addition, niacin can lower LDL cholesterol and increase HDL cholesterol; both have beneficial effects.

Statin (also referred to as HMG-CoA reductase inhibitor): This class of lipid-lowering drugs, introduced in the late 1980s, is fast becoming the most widely prescribed class of drugs to lower cholesterol. Fluvastatin (Lescol®), lovastatin (Mevacor®), simvastatin (Zocor®), pravastatin (Pravachol®), atorvastatin (Lipitor®), and cerivastatin are exemplary statins. Statins work directly in the liver to inhibit a key enzyme involved in the biosynthesis of cholesterol; statins effectively deplete cholesterol in the liver cells and cause the cells to remove cholesterol from circulating blood. Depending on the dose, statins can reduce LDL cholesterol by up to 40 percent. Statins may also help the body to reabsorb cholesterol from plaques, and thereby serving to slowly unclog the blood vessels. Statins reduce inflammation around the plaques, which helps to stabilize the plaques and reduce the chances of rupture and blockage of the affected artery. Statin is the only type of lipid-lowering drug proven to reduce the risk of death from cardiovascular disease. Along with niacin, statin has also been proven to reduce the risk of having a second heart attack.

Meso-formyl porphyrins, meso-acrylate porphyrins, purpurins and benzochlorins and mono-formylated tetrapyrrolic may have a healing effect on calcified and vulnerable plaque. In one aspect of the present invention, the drug delivery device comprises meso-formyl porphyrin, meso-acrylate porphyrin, purpurin, benzochlorin, mono-formylated tetrapyrrolic, or a combination thereof.

Tamoxifen is a drug widely used for the treatment of breast cancer. In one aspect of the present invention, the drug delivery device comprises tamoxifen.

Other pharmacologically active agents suitable for use herein are as follows:
Anti-diarrheals, such as diphenoxylate, loperamide and hyoscyamine;
Anti-hypertensives, such as clonidine, prazosin, debrisoquine, diazoxide, guanethidine, reserpine, and trimethaphan;
Calcium channel blockers, such as diltiazem, and nitrendipine;
Anti-arrhyrthmics, such as mexiletene and quinidine;
Anti-angina agents, such as glyceryl trinitrate, erythrityl tetranitrate, pentaerythritol tetranitrate, mannitol hexanitrate, perhexylene, and nicorandil;
Beta-adrenergic blocking agents, such as alprenolol, bupranolol, carteolol, nadolol, nadoxolol, oxprenolol, pindolol, timolol and timolol maleate;
Cardiotonic glycosides, such as cardiac glycosides and theophylline derivatives;
Adrenergic stimulants, such as adrenaline, ephedrine, fenoterol, isoprenaline, orciprenaline, rimeterol, salbutamol, salmeterol, terbutaline, dobutamine, phenylephrine, phenylpropanolamine, and pseudoephedrine;
Vasodilators, such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol;
Anti-migraine preparations, such as ergotanmine, dihydroergotamine, methysergide, pizotifen and sumatriptan;
Anti-coagulants and thrombolytic agents, such as dicoumarol, low molecular weight heparins such as enoxaparin, and active derivatives of streptokinase;
Hemostatic agents, such as aprotinin, tranexarnic acid and protamine;
Analgesics and anti-pyretics including the opioid analgesics, such as buprenorphine, dextromoramide, dextropropoxyphene, fentanyl, alfentanil, sufentanil, hydromorphone, methadone, morphine, oxycodone, papavereturn, pentazocine, pethidine, phenopefidine, codeine dihydrocodeine; paracetamol, and phenazone;
Neurotoxins, such as capsaicin;
Hypnotics and sedatives, such as the barbiturates amylobarbitone, butobarbitone and pentobarbitone and other hypnotics and sedatives such as chloral hydrate, chlormethiazole, hydroxyzine and meprobamate;
Anti-anxiety agents, such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam;
Neuroleptic and anti-psychotic drugs, such as the phenothiazines, chlorpromazine, flupbenazine, pericyazine, perphenazine, promazine, thiopropazate, thioridazine, trifluoperazine; and butyrophenone, droperidol and haloperidol; and other antipsychotic drugs, such as pimozide, thiothixene and lithium;
Anti-depressants, such as tricyclic antidepressants (such as amitryptyline, clomipramine, desipramine, dothiepin, doxepin, imipramine, nortriptyline, opipramol, protriptyline and trimipramine), tetracyclic antidepressants (such as mianserin), monoamine oxidase inhibitors (such as isocarboxazid, phenelizine, tranylcypromine and moclobemide), and selective serotonin re-uptake inhibitors (such as fluoxetine, paroxetine, citalopram, fluvoxamine and sertraline);
CNS stimulants, such as caffeine and 3-(2-aminobutyl) indole;
Anti-Alzheimer's agents, such as tacrine;
Anti-Parkinson's agents, such as amantadine, benserazide, carbidopa, levodopa, benztropine, bipefiden, benzhexyl, procyclidine and dopamine-2 agonists;
Anti-convulsants, such as phenyloin, valproic acid, primidone, phenobarbitone, methylphenobarbitone and carbamazepine, ethosuximide, methsuximide, phensuximide, sulthiame and clonazepam;
Anti-emetics and anti-nauseants, such as the phenothiazines prochloperazine, thiethylperazine and 5HT-3 receptor antagonists, such as ondansetron and granisetron, as well as dimenhydrinate, diphenhydramine, metoclopramide, domperidone, hyoscine, hyoscine hydrobromide, hyoscine hydrochloride, clebopride and brompride;
Non-steroidal anti-inflammatory agents, including their racemic mixtures or individual enantiomers where applicable, preferably formulated in combination with dermal penetration enhancers, such as ibuprofen, flurbiprofen, ketoprofen, aclofenac, diclofenac, aloxiprin, aproxen, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, atninopyrine, antipyrine, oxyphenbutazone, apazone, cintazone, flufenamic acid, clonixerl, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate;
Anti-rheumatoid agents, such as penicillamine, aurothioglucose, sodium aurothiomalate, methotrexate and auranofin;
Muscle relaxants, such as baclofen, diazepam, cyclobenzaprine hydrochloride, dantrolene, methocarbamol, orphenadrine and quinine;
Agents used in gout and hyperuricaemia, such as allopurinol, colchicine, probenecid and sulphinpyrazone;
Progesterone and other progestagens, such as allyloestrenol, dydrgesterone, lynoestrenol, norgestrel, norethyndrel, norethisterone, norethisterone acetate, gestodene, levonorgestrel, medroxyprogesterone and megestrol;
Androgens and anabolic agents, such as testosterone, methyltestosterone, clostebol acetate, drostanolone, furazabol, nandrolone oxandrolone, stanozolol, trenbolone acetate, dihydro-testostero 17-(a-methyl-19-noriestosterone and fluoxymesterone;
Anti-androgens, such as cyproterone acetate and danazol;
Oestrogens, such as oestradiol, oestriol, oestrone, ethinyloestradiol, mestranol, stilboestrol, dienoestrol, epioestriol, estropipate and zeranol;
Anti-oestrogens, such as epitiostanol and the aromatase inhibitors, exemestane and 4-hydroxy-androstenedione and its derivatives;
5-alpha reductase inhibitors, such as finastride, turosteride, LY-191704 and MK-306-1;
Cortico steroids, such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, and triamcinolone acetonide;
Glycosylated proteins, proteoglycans, glycosaminoglycans such as chondroitin sulfate; chitin, acetyl-glucosamine, and hyaluronic acid;
Complex carbohydrates, such as glucans;
Steroidal anti-inflammatory agents, such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlofisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazoll;
Pituitary hormones and their active derivatives or analogs, such as corticotrophin, thyrotropin, follicle stimulating hormone (FSH), luteinising hormone (LH) and gonadotrophin releasing hormone (GnRH);
Hypoglycemic agents, such as insulin, chlorpropamide, glibenclamide, gliclazide, glipizide, tolazamide, tolbutamide and metformin;
Thyroid hormones, such as calcitonin, thyroxine and liothyronine, and anti-thyroid agents such as carbimazole and propylthiouracil;
Other miscellaneous hormone agents, such as octreotide;
Pituitary inhibitors, such as bromocriptine;
Ovulation inducers, such as clomiphene;
Diuretics, such as thiazides, related diuretics and loop diuretics, bendrofluazide, chlorthalidone, cyclopenthiazide, hydrochlorothiazide, indapamide, mefruside, methycholthiazide, metolazone, quinethazone, bumetanide, ethacrynic acid and frusemide and potassium sparing diuretics, spironolactone, amiloride and triamterene;
Anti-diuretics, such as desmopressin, lypressin and vasopressin including their active derivatives or analogs;
Obstetric drugs including agents acting on the uterus, such as ergometfine, oxytocin and gemeprost;
Prostaglandins, such as alprostadil (PGEI), prostacyclin (PG12), dinoprost (prostaglandin F2-alpha) and misoprostol;
Anti-microbials, including the cephalospofins such as cephalexin, cefoxytin and cephalothin;
Penicillins, such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin;
Tetracyclines, such as minocycline, chlortetracycline, tetracycline, demeclocycline, doxycycline, methacycline and oxytetracycline and other tetracycline-type antibiotics;
Aminoglycoides, such as amikacin, gentamicin, kanamycin, neomycin, netilmicin and tobramycin;
Anti-fungals, such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyfithione;
Quinolones, such as nalidixic acid, cinoxacin, ciprofloxacin, enoxacin and norfloxacin;
Sulphonamides, such as phthalysulphthiazole, sulfadoxine, sulphadiazine, sulphamethizole and sulphamethoxazole;
Sulphones, such as dapsone;
Antibiotics, such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonarn, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroan-tine compounds; and benzoylperoxide;
Anti-tuberculosis drugs, such as ethambutol, isoniazid, pyrazinamide, rifampicin and clofazimine;
Anti-malarial agents, such as primaquine, pyrimethamine, chloroquine, hydroxychloroquine, quinine, mefloquine and halofantrine;
Anti-viral agents, such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine;
Anti-helmintic agents, such as mebendazole, thiabendazole, niclosamide, praziquantel, pyrantel embonate and diethylcarbamazine;
Cytotoxic agents, such as plicamycin, cyclophosphamide, dacarbazine, fluorouracil and its prodrugs, methotrexate, procarbazine, 6-mercaptopurine and mucophenolic acid;
Anorectic and weight reducing agents, including dexfenfluramine, fenfluramine, diethylpropion, mazindol and phentermine;
Agents used in hypercalcaemia, such as calcitriol, dihydrotachysterol and their active derivatives or analogs;
Anti-tussive drugs, such as ethylmorphine, dextromethorphan and pholcodine;
Expectorants, such as carbolcysteine, bromhexine, emetine, quanifesin, ipecacuanha and saponins;
Decongestants, such as phenylephrine, phenylpropanolamine and pseudoephedrine;
Bronchospasm relaxants, such as ephedrine, fenoterol, orciprenaline, rimiterol, salbutamol, sodium cromoglycate, cromoglycic acid and its prodrugs, terbutaline, ipratropium bromide, salmeterol and theophylline and theophylline derivatives;
Anti-histamines, such as meclozine, cyclizine, chlorcyclizine, hydroxyzine, brompheniramine, chlorpheniramine, clemastine, cyproheptadine, dexchlorpheniramine, diphenhydramine, diphenylamine, doxylatnine, mebhydrolin, pheniramine, tripolidine, azatadine, diphenylpyraline, methdilazine, terfenadine, astemizole, loratidine and cetirizine;
Local anaesthetics, such as bupivacaine, amethocaine, lignocaine, lidocaine, cinchocaine, dibucaine, mepivacaine, prilocaine, etidocaine, veratridine (specific c-fiber blocker) and procaine;
Stratum corneum lipids, such as ceramides, cholesterol and free fatty acids, for improved skin barrier repair;
Neuromuscular blocking agents, such as suxamethonium, alcuronium, pancuronium, atracurium, gallamine, tubocurarine and vecuronium;
Smoking cessation agents, such as nicotine, bupropion and ibogaine;
Insecticides and other pesticides which are suitable for local application;
Dermatological agents, such as vitamins A, C, B1, B2, B6, B12, and E, vitamin E acetate and vitamin E sorbate;
Allergens for desensitization, such as house, dust or mite allergens;
Nutritional agents and neutraceuticals, such as vitamins, essential amino acids and fats;
Acromolecular pharmacologically active agents, such as proteins, enzymes, peptides, polysaccharides (such as cellulose, amylose, dextran, chitin), nucleic acids, cells, tissues, and the like; and
Keratolytics, such as the alpha-hydroxy acids, glycolic acid and salicylic acid.

The device of the present invention may comprise a pharmaceutical composition comprising acarbose; acyclovir; acetyl cysteine; acetylcholine chloride; alatrofloxacin; alendronate; alglucerase; amantadine hydrochloride; ambenomium; amifostine; amiloride hydrochloride; aminocaproic acid; amphotericin B; antihemophilic, factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotoxime; cefoxitin sodium; ceftizoxime; ceftriaxone; cefuroxime axetil; cephalexin; cephapirin sodium; cholera vaccine; chrionic gonadotropin; cidofovir; cisplatin; cladribine; clidinium bromide; clindamycin and clindamycin derivatives; ciprofloxacin; clondronate; colistimethate sodium; colistin sulfate; cortocotropin; cosyntropin; cromalyn sodium; cytarabine; daltaperin sodium; danaproid; deforoxamine; denileukin diftitox; desmopressin; diatrizoate megluamine and diatrizoate sodium; dicyclomine; didanosine; dirithromycin; dopamine hydrochloride; dornase alpha; doxacurium chloride; doxorubicin; editronate disodium; elanaprilat; enkephalin; enoxacin; enoxaprin sodium; ephedrine; epinephrine; epoetin alpha; erythromycin; esmol hydrochloride; factor IX; famiciclovir; fludarabine; fluoxetine; foscarnet sodium; ganciclovir; granulocyte colony stimulating factor; granulocyte-macrophage stimulating factor; growth hormones-recombinant human; growth hormone-bovine; gentamycin; glucagon; glycopyrolate; gonadotropin releasing hormone and synthetic analogs thereof; GnRH; gonadorelin; grepafloxacin; hemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; isofosfamide; japanese encephalitis virus vaccine; lamivudine; leucovorin calcium; leuprolide acetate; levofloxacin; lincomycin and lincomycin derivatives; lobucavir; lomefloxacin; loracarbef; mannitol; measles virus vaccine; meningococcal vaccine; menotropins; mephenzolate bromide; mesalmine; methanamine; methotrexate; methscopolamine; metformin hydrochloride; metroprolol; mezocillin sodium; mivacurium chloride; mumps viral vaccine; nedocromil sodium; neostigmine bromide; neostigmine methyl sulfate; neutontin; norfloxacin; octreotide acetate; ofloxacin; olpadronate; oxytocin; pamidronate disodium; pancuronium bromide; paroxetine; pefloxacin; pentamindine isethionate; pentostatin; pentoxifylline; periciclovir; pentagastrin; phentolamine mesylate; phenylalanine; physostigmine salicylate; plague vaccine; piperacillin sodium; platelet derived growth factor-human; pneumococcal vaccine polyvalent; poliovirus vaccine inactivated; poliovirus vaccine live (OPV); polymixin B sulfate; pralidoxine chloride; pramlintide; pregabalin; propofenone; propenthaline bromide; pyridostigmine bromide; rabies vaccine; residronate; ribavarin; rimantadine hydrochloride; rotavirus vaccine; salmetrol xinafoate; sincalide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; typhoid vaccine live; urea; urokinase; vancomycin; valaciclovir; valsartan; varicella virus vaccine live; vasopressin and vasopressin derivatives; vecoronium bromide; vinblastin; vincristine; vinorelbine; vitamin B12; warfarin sodium; yellow fever vaccine; zalcitabine; zanamavir; zolandronate; zidovudine; pharmaceutically acceptable salts, isomers and derivatives thereof; or combinations thereof.

Additional pharmacologically active agents suitable for use herein include angiogenic factors, growth factors, inotropic agents, anti-atherogenic agents, anti-coagulants (those not listed in Table I), anti-arrhythmic agents (those not listed in Table I), sympathomimetic agents, phosphodiesterase inhibitors, antineoplastic agents, and steroids.

The drug(s) of the present device preferably elute over a time period, for example, of up to one day, one week, one month, one year, or ten years.

The device of the present invention is useful for local delivery of drugs to treat cardiovascular or vascular diseases, such as plaques or stenosis. The present device may also be used as an alternative over stents, for patients who comprise multiple stents in the treated vessel. The device of the present invention is also suitable for use with patients who have already undergone vascular procedures, such as a PTCA, or who are classified as high-risk patients due to their family history, their high LDL (low density lipoprotein) or CRP (C-Reactive Protein) levels.

In one aspect, the present device comprises different areas, with each area comprising drug(s) that is different from the drug(s) contained in other areas; in another aspect, the device comprises different areas with each area comprising the same drug(s) but in different concentrations from the drug concentrations in other areas. In one aspect, the present device comprises small depots for containing liquid or gel-based drugs, the depots open as the matrix material vanishes by elution, whereby delivering the drug to the targeted location, e.g., the bloodstream.

### EXAMPLES

Having generally described the invention, a more complete understanding thereof may be obtained by reference to the following examples that are provided for purposes of illustration only and do not limit the invention.

### Example 1

### Device Having an RLS Configuration

A device having an RLS configuration comprises an outer layer of polymeric matrix, which contains a drug of a high concentration that elutes very quickly, and an inner core of polymeric matrix containing a drug, which elutes slowly over a long period of time. This RLS is useful for treating a stenosis proximal downstream to the RLS, and thus preventing the vessel part from restenosis.

### Example 2

### Device Having an RLS Configuration and Comprising Two Different Drugs

A device having an RLS configuration comprises only one material matrix, which contains two different drugs with different wash-out-characteristics. First drug elutes very quickly, while the second drug elutes slowly over time. The second drug may only elute while the matrix material of the RLS slowly elutes over time, while the first drug washes out of the matrix material quickly. This RLS is also useful for treating a stenosis proximal downstream to the RLS, and thus preventing the vessel part from restenosis.

### Example 3

### Device Having an FLS Configuration

A device having an FLS configuration comprising a ring-shaped holding structure and a plurality of flags is deployed in a cardiovascular vessel. The holding structure and the flags of the FLS comprise a metal matrix material. The plurality of flags comprises atropine. This FLS is useful for treating bradycardia and heart blockage.

### Example 4

### Device Having a PLS Configuration

A device having an PLS configuration comprising a polymeric matrix that comprises celecoxib is deployed, via a balloon catheter, in a renal artery. This PLS is useful for treating inflammation in the kidneys.

While the above description of the invention has been presented in terms of a human subject (patient), it is appreciated that the invention may also be applicable to treating other subjects, such as non-human mammals.

As noted above, the present invention is applicable to implantable devices designed for releasing a drug to treat or prevent cardiovascular or vascular diseases, or diseases that may be attributable to inflammation, and methods related thereto. The present invention should not be considered limited to the particular aspects described above, but rather should be understood to cover all aspects of the invention as fairly set out in the appended claims. Various modifications, equivalent processes, as well as numerous structures to which the present invention may be applicable will be readily apparent to those skilled in the art to which the present invention is directed upon review of the present specification. The claims are intended to cover such modifications and devices.

We claim:

1. A device for implanting in the vasculature or cardiovasculature for treating or preventing a disease, comprising:
a) a biodegradable matrix material capable of dissolving upon contact with blood,
b) at least one drug capable of being released into the blood stream as the biodegradable matrix material degrades,
said device comprising a holding structure and at least one flag, said at least one flag being attached to said holding structure at one portion and the remaining portion of said at least one flag floating or lying in the bloodstream; and
said device being capable of degrading gradually and completely as the biodegradable matrix material degrades.

2. A device according to claim 1, said holding structure being a ring-shaped structure.

3. A device according to claim 1, said at least one flag comprising fibers, woven tissue, strings, sheets, or any combination thereof.

4. A device according to claim 1, said at least one flag having elastic, twisted, unwoven, woven, or tapered construction.

5. A device according to claim 1, said biodegradable matrix material comprising a polymeric material, a non-polymeric organic material a metallic material, or any combination thereof.

6. A device according to claim 1, said biodegradable matrix material comprising an epoxy, polyester, acrylic, polyanhydride, polyurethane, poly(tetrafluoroethylene), polycaprolactone, polyethylene oxide, polyethylene glycol, poly(vinyl chloride), polylactic acid, polypropylene oxide, poly(alkylene)glycol, polyoxyethylene, sebacic acid, polyvinyl alcohol, 2-hydroxyethyl methacrylate, polymethyl methacrylate, 1,3-bis(carboxyphenoxy)propane, phosphatidylcholine, triglyceride, polyhydroxybutyrate, polyhydroxyvalerate, poly(ethylene oxide), poly ortho ester, poly (amino acid), polycynoacrylate, polyphophazene, polysulfone, polyamine, poly (amido amine), siloxane-based elastomer, styrene, flexible fluoropolymer, vinyl pyrrolidone, cellulose acetate dibutyrate, lipid, or any combination thereof.

7. A device according to claim 1, said biodegradable matrix material comprising a naturally occurring protein, a synthetic protein, or a combination thereof.

8. A device according to claim 1, said biodegradable matrix material comprising a shape-memory effect material.

9. A device according to claim 1, comprising at least one depot for storing said at least one drug, said at least one depot opening and releasing said at least one drug as the biodegradable matrix material degrades.

10. A device according to claim 1, said at least one drug comprising a resin, fibrate, niacin, statin, paclitaxel, adenosine, spironolactone, alteplace, amlodipine, amiodarone, anistreplase, aspirin, atenolol, atropine, abciximab, captopril, carvedilol, celecoxib, chlorothiazide, cholestyramine, clofibrate, clopidrogel, digoxin, dipyridamole, disopyramide, dobutamine, dofetilide, dopamine, enalapril, epinephrine, felodipine, flecamide, furosemide, losartan, lovastatin, metoprolol, minoxidil, nifedipine, nimodipine, pravastatin, procainamide, popranolol, protamine, simvastatin, sotalol, streptokinase, ticlodipine, urokinase, verapamil, warfarin, or any combination thereof.

11. A device according to claim 1, said at least one drug comprising an anti-inflammatory agent.

12. A device according to claim 1, comprising a drug releasing agent.

13. A device according to claim 1, said holding structure, said at least one flag or both comprising a plurality of areas, each area of said plurality of areas comprising a drug, wherein the drug in at least one area of said plurality of areas being the same drug as in other areas of said plurality of areas, or the drug in at least one area of said plurality of areas being the same drug having a different concentration from the same drug in other areas of said plurality of areas, or the drug in at least one area of said plurality of areas being different from the drug in other areas of said plurality of areas.

14. A device according to claim 1, said biodegradable matrix material comprising one or more particles, said at least one drug being coated onto or incorporated into the one or more particles.

15. A device according to claim 14, said one or more particles comprising iron oxide (Fe₃O₄), titaniumoxide (TiO₂), magnesium oxide, aluminum oxide, zirconium oxide, palladium oxide, titanium, titanium alloy, iron-based alloy, nickel-based alloy, zinc-based alloy, aluminum-based alloy, molybdenum-based alloy, vanadium-based alloy, cobalt-based alloy, palladium-cobalt, zirconia, or any combination thereof.

16. A device according to claim 14, said one or more particles being capable of changing the contrast in a radiological imaging system.

17. A device according to claim 16, said one or more particles comprising iron oxide (Fe₃O₄), titanium, titanium alloy, titaniumoxide (TiO₂), magnesiumoxide, palladiumoxide, palladiumcobalt, ⁹⁰Y, ¹³³Xe, ^{81m}Kr, ¹¹¹In, ^{133m}In, ²⁰¹Th, or any combination thereof.

18. A device according to claim 1, comprising Zyn-Linkers.

19. A device according to claim 1, comprising a binder.
